(19) United States
(12) Patent Application Publication
(10) Pub. No.: US 2025/0376652 A1
(43) Pub. Date: Dec. 11, 2025

(54) APPARATUS AND METHODS FOR MAGNETICALLY COUPLING BIOREACTOR DRIVE HEADS

(71) Applicant: Global Life Sciences Solutions USA LLC, Marlborough, MA (US)

(72) Inventors: Nagaraj RAO, Bengaluru (IN); Kandakumar MURUGESAN, Bengaluru (IN); Saravanan BALAKRISHNAN, Bengaluru (IN)

(73) Assignee: Global Life Sciences Solutions USA LLC, Marlborough, MA (US)

(21) Appl. No.: 18/875,544

(22) PCT Filed: Jun. 12, 2023
(86) PCT No.: PCT/US2023/025040

§ 371 Date: Dec. 16, 2024

(30) Foreign Application Priority Data
Jun. 22, 2022 (IN) 202211035832

(51) Int. Cl. C12M 1/06; C12M 1/00

(52) U.S. Cl. CPC C12M 27/02; C12M 23/14

(57) ABSTRACT

A vessel including an interior volume configured to contain a liquid and a magnetically driven impeller located within the interior volume. The impeller including a rotatable base portion with a blade, a rotatable shaft, and a ferrous connector. The impeller may be coupled to an external motor and rotated to agitate liquid in the interior volume, via a magnetic bond between the ferrous connector and a selectively magnetizable drive head connector of the external motor. The ferrous connector is not a permanent magnet.

### CROSS-REFERENCE TO RELATED APPLICATIONS

This application is a national stage of International Application No. PCT/US2023/025040 filed on Jun. 12, 2023, which claims the benefit of India application Ser. No. 202211035832, filed on Jun. 22, 2022, all of which are hereby incorporated by reference herein in their entireties.

### BACKGROUND

### Technical Field

Embodiments of the invention relate generally to bioreactor systems and methods and, more particularly, to an apparatus and method for selectively magnetically coupling drive heads to impellers in stirred tank bioreactor and mixer systems.

### Discussion of Art

Increasingly, in the biopharmaceutical industry, single use or disposable containers are used. Such containers can be flexible or collapsible plastic vessels or bags that are supported by an outer rigid structure such as a stainless-steel shell or housing. Use of sterilized disposable bags eliminates time-consuming step of cleaning of the housing and reduces the chance of contamination. The bag may be positioned within the rigid housing and filled with the desired fluid for mixing. An agitator assembly disposed within the bag is used to mix the fluid. Existing agitators are either top-driven (having a shaft that extends downwardly into the bag, on which one or more impellers are mounted) or bottom-driven (having an impeller disposed in the bottom of the bag that is driven by a magnetic drive system or motor positioned outside the bag and/or vessel). Most magnetic agitator systems include a drive motor with a rotating magnetic drive head outside of the bag and a rotating magnetic agitator (also referred to in this context as the “impeller”) within the bag. The movement of the magnetic drive head enables torque transfer and thus rotation of the magnetic agitator allowing the agitator to mix a fluid within the vessel.

In current designs, however, impellers containing costly permanent magnets are located within the disposable bag and are discarded with the bag after a single use. Moreover, in known systems, there is a gap between the magnets of the drive head and the permanent magnets of the impeller to accommodate design tolerances. This magnetic gap may reduce holding torque and rotational speed of the drive motor. Further, increasing torque and rotational speed of known magnetic drive heads and impellers is challenging as increasing the number and size of the magnets may not be possible. Known magnetic drive heads require multi-component lifting equipment to couple the drive head with an impeller for use. Finally, decoupling of the magnets of the drive head and the permanent magnets of the impeller is also a difficult process, requiring significant forces and/or additional equipment.

In view of the above, there is a need for an impeller and drive head that are selectively magnetically couplable without the need for an impeller with permanent magnets or expensive lift equipment, and that do not include a significant magnetic gap when coupled.

### BRIEF DESCRIPTION

In an embodiment, a vessel includes an interior volume configured to contain a liquid and a magnetically driven impeller located within the interior volume. The impeller includes a rotatable base portion with at least one blade, a rotatable shaft, and at least one ferrous connector. The impeller may be coupled to an external motor and rotated to agitate liquid in the interior volume via a magnetic bond between the ferrous connector and a selectively magnetizable drive head connector of the external motor. The ferrous connector is not a permanent magnet.

In another embodiment of the invention, a method for coupling an impeller of a vessel to a drive motor is provided. The method includes placing a drive head, which is in a demagnetized state, of the drive motor in proximity to an impeller, which includes a ferrous impeller connector, that is located within an interior volume of the vessel. Then, the method includes magnetizing the drive head to create a magnetic bond between a drive head connector of the drive motor and the ferrous impeller connector.

In yet another embodiment, a method of agitating fluid in a vessel is provided. The method includes rotating a drive head of a drive motor to rotate an impeller located within an interior volume of the vessel. Rotation of the impeller is accomplished via a magnetic bond between a selectively magnetizable drive head connector of the drive motor and a ferrous connector of the impeller.

In an additional embodiment, a method of decoupling an impeller of a vessel from a drive motor is provided. The method includes demagnetizing a drive head of the drive motor to remove a magnetic bond between a drive head connector of the drive motor and a ferrous impeller connector of an impeller located within an interior volume of the vessel. The drive motor may then be moved away from the impeller.

### DRAWINGS

The present invention will be better understood from reading the following description of non-limiting embodiments, with reference to the attached drawings, wherein below:

FIG. 1 is a front elevational view of a bioreactor system.

FIG. 2 is a front elevational view of a prior art impeller compatible with the bioreactor system of FIG. 1.

FIG. 3 is a side sectional view of the prior art impeller of FIG. 2.

FIG. 4 is an enlarged side sectional view of the portion of the prior art impeller of FIG. 2 within rectangle A.

FIG. 5 is an exploded elevational view of an impeller, a ferrous connector, and a drive head connector, according to an embodiment of the present invention.

FIG. 6 is the exploded elevational view of FIG. 5 with the impeller, ferrous connector, and drive head connector depicted in phantom, according to an embodiment of the present invention.

FIG. 7 is a side sectional view of the impeller, ferrous connector, and drive head connector of FIGS. 5 and 6, according to an embodiment of the present invention.

FIG. 8 is the portion of the impeller, ferrous connector, and drive head of FIG. 7 within rectangle B in a magnetized state, according to an embodiment of the present invention.

FIG. 9 is the portion of the impeller, ferrous connector, and drive head of FIG. 7 within rectangle B in a demagnetized state, according to an embodiment of the present invention.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts.

As used herein, the term “flexible” or “collapsible” refers to a structure or material that is pliable, or capable of being bent without breaking, and may also refer to a material that is compressible or expandable. An example of a flexible structure is a bag formed of polyethylene film. The terms “rigid” and “semi-rigid” are used herein interchangeably to describe structures that are “non-collapsible,” that is to say structures that do not fold, collapse, or otherwise deform under normal forces to substantially reduce their elongate dimension. Depending on the context, “semi-rigid” can also denote a structure that is more flexible than a “rigid” element, e.g., a bendable tube or conduit, but still one that does not collapse longitudinally under normal conditions and forces.

A “vessel,” as the term is used herein, means a flexible bag, a flexible container, a semi-rigid container, or a rigid container, as the case may be. The term “vessel” as used herein is intended to encompass bioreactor vessels having a wall or a portion of a wall that is flexible or semi-rigid, single use flexible bags, as well as other containers or conduits commonly used in biological or biochemical processing, including, for example, cell culture/purification systems, mixing systems, media/buffer preparation systems, and filtration/purification systems.

As used herein, the term “bag” means a flexible or semi-rigid container or vessel used, for example, as a bioreactor or mixer for the contents within. In this regard, embodiments of the invention are suitable for use with bioreactors, mixers, and other devices or systems with in-vessel impellers and external drive motors.

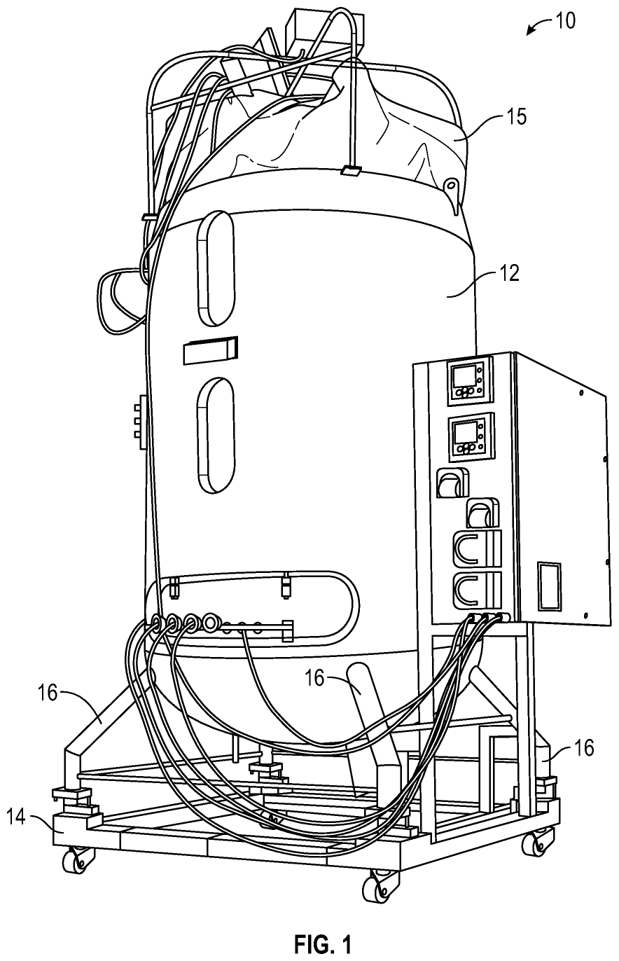

With reference to FIG. 1, a bioreactor system 10 suitable for use with embodiments of the invention is illustrated. The bioreactor system 10 includes a generally rigid bioreactor housing 12 mounted atop a base 14 having a plurality of legs 16. The housing 12 may be formed, for example, from stainless steel, polymers, composites, glass, or other metals, and may be cylindrical in shape, although other shapes may also be utilized without departing from the broader aspects of the invention. In certain embodiments, the housing 12 may be a substantially rectangular mixer housing.

As shown, a single-use, flexible vessel or bag 15 is disposed within the housing 12. As mentioned, the housing 12 can be any size (or shape) as long as it is capable of supporting a single-use flexible bioreactor bag 15. For example, according to one embodiment, the housing 12 is capable of accepting and supporting a 10-2000L flexible or collapsible bioprocess bag assembly 20.

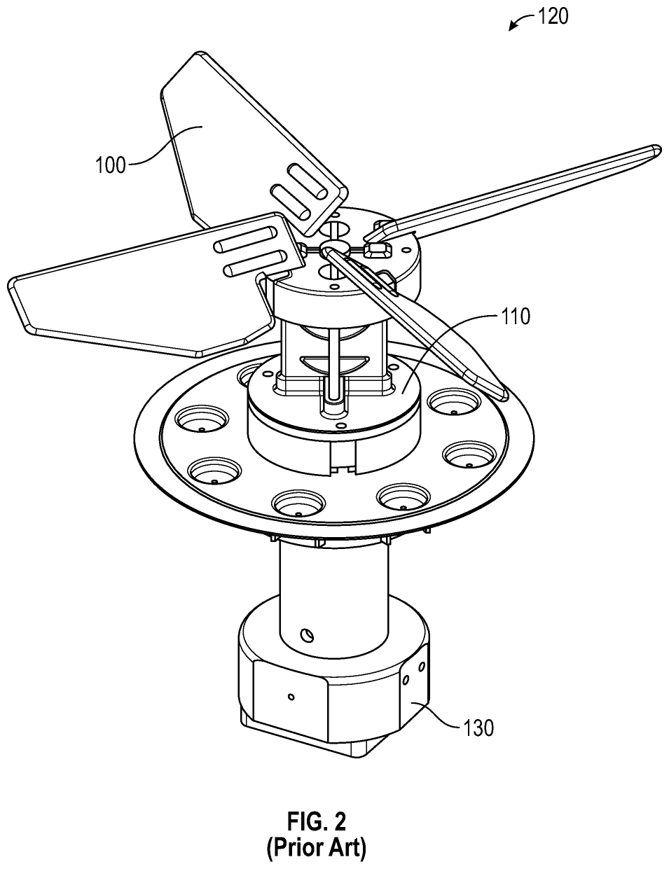

With specific reference to FIGS. 2 and 3, a known impeller 100 commonly used with the bioreactor system 10 is depicted. The flexible bag 15 contains the impeller 100 attached to a magnetic hub 110 at the bottom of the inside of the bag. Together, the impeller 100 and hub 110 (and in some embodiments, the impeller plate) form an impeller assembly 120. A magnetic drive 130 external to the housing 12 provides the motive force for rotating the magnetic hub 110 and impeller 100 to agitate the contents of the flexible bag 15. The magnetic hub 110 has permanent impeller magnets 112 and the magnetic drive 130 includes permanent drive magnets 132.

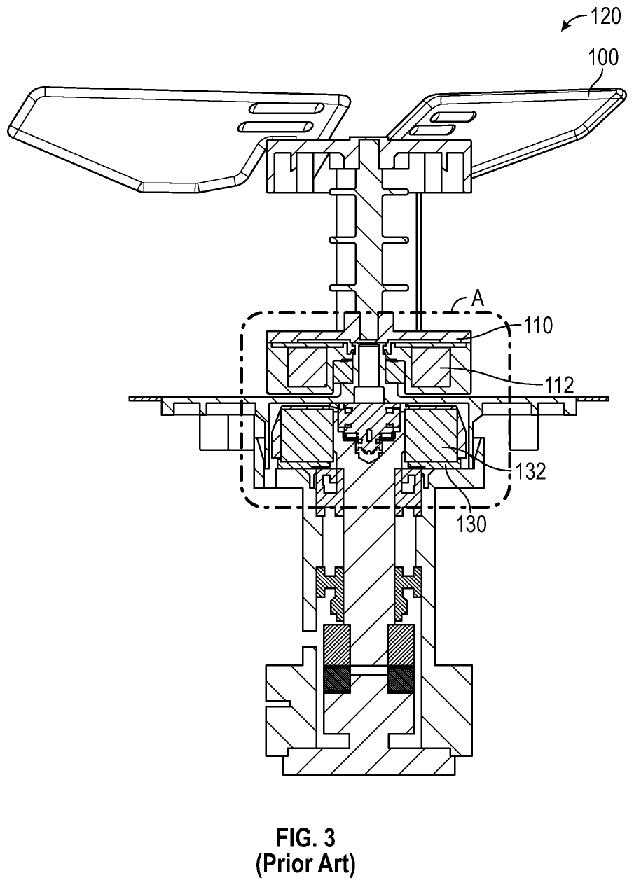

These known impellers 100 are limited as they require permanent magnets in both the magnetic hub 110 and the magnetic drive 130, which leads to several potential design issues. First, the number and size of the required permanent magnets may be inadequate to support the required torque and impeller agitation speed in certain applications. Increasing the number or size of the permanent magnets may not be feasible, e.g., due to size or cost constraints. Second, known drive heads are coupled and decoupled with the impeller 100 using lifting equipment that required significant forces and include numerous parts which contribute to a high cost of the magnetic drive 130. Third, permanent magnets 112 within the magnetic hub 110 are discarded with the other consumables. As will be appreciated, these permanent magnets 112 are costly to replace and can be costly to properly dispose of. Fourth, the vertical movement of the drive head requires a significant gap (between the magnets 112 of the magnetic hub 110 and the magnets 132 of the magnetic drive 130 (as depicted in FIGS. 3 and 4). The gap G is necessary to accommodate the tolerances of the structural elements that house and surround the magnets 112, 132 within the magnetic hub 110 and the magnetic drive 130, respectively.

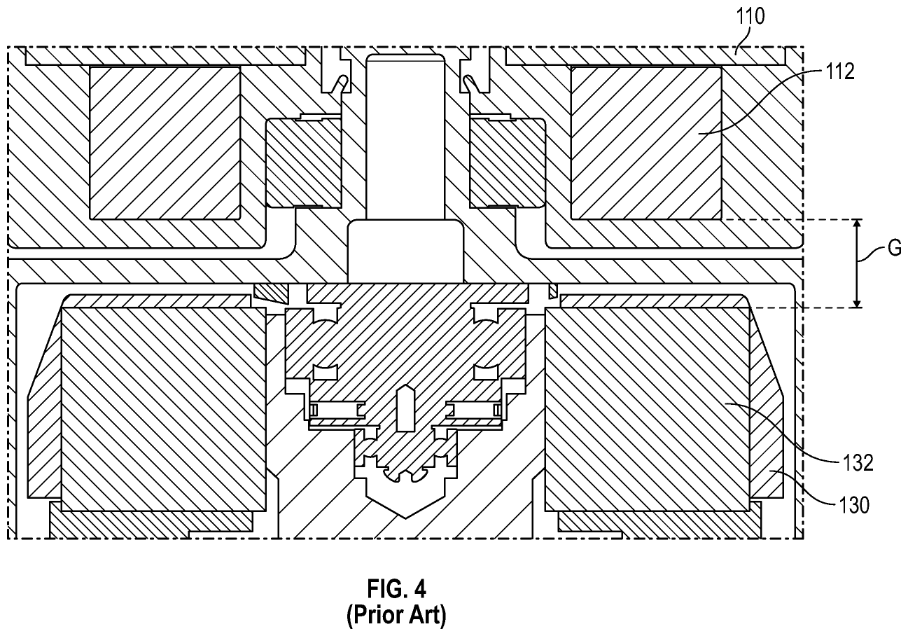

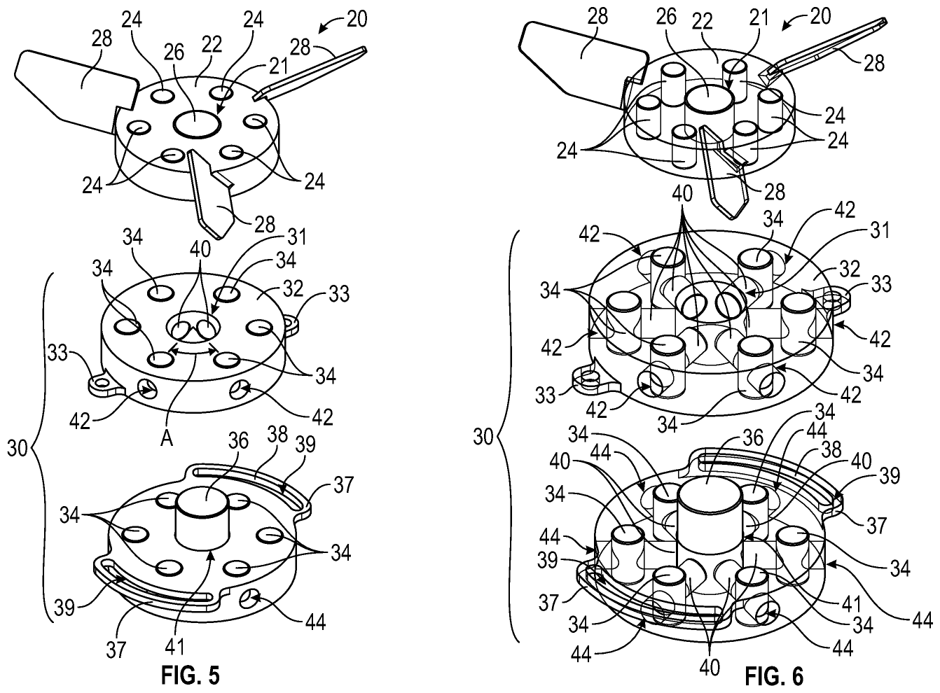

Referring back to FIG. 1, the bag 15 includes an interior volume configured to contain a liquid and a magnetically driven impeller 20 located within the interior volume. As illustrated in FIGS. 5 and 6, the impeller 20 includes a rotatable base portion 22 with at least one blade 28, a rotatable shaft 26, and at least one ferrous connector 24. The impeller 20 may be coupled to an external drive motor and rotated to agitate liquid in the interior volume via a magnetic bond between the ferrous connector 24 of impeller 20 and a ferrous connector 34 of a selectively magnetizable drive head 30 of the drive motor. In embodiments, the ferrous connectors 24, 34 are not permanent magnets.

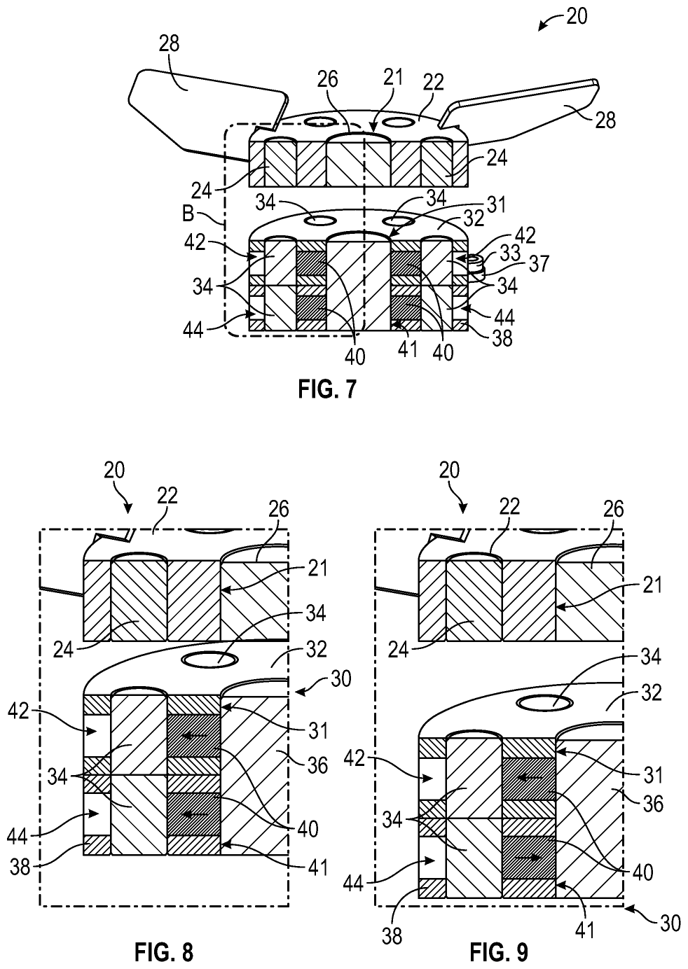

In the embodiments depicted in FIGS. 5-9, the impeller 20 does not contain any permanent magnets. In these embodiments, the impeller 20 includes a plurality of ferrous connectors 24 in the base portion 22 of the impeller 20. The ferrous connectors 24 are spaced apart about a central aperture that accommodates the rotating shaft 26. In the embodiment depicted in FIGS. 5-9, six (6) ferrous connectors 24 are equidistantly spaced about the rotating shaft 26. A different number and/or a different arrangement of the ferrous connectors 24 does not depart from the invention disclosed herein. In one embodiment, the ferrous connector 24 is made of steel, but other ferromagnetic materials, including but not limited to Iron, Cobalt, Nickel, and alloys thereof do not depart from the scope of the invention. In alternative embodiments, the impeller 20 may include one or more permanent magnets (ideally fewer than in known impellers) in addition to ferrous connectors 24.

In the embodiment depicted in FIGS. 5-9, the impeller 20 includes three blades 28. As will be readily appreciated, however, the impeller 20 may include greater or fewer than three blades. In certain embodiments, it may be possible for the impeller 20 to include multiple tiers or rows of blades. The blades 28 may have a variety of sizes, shapes, positions, and angles without departing from the invention.

In embodiments, the impeller 20 is configured for use with a drive head that may be selectively magnetizable. Such drive heads may be mechanically magnetizable, such as the drive head 30 depicted in FIGS. 5-9 and described below, or, in other embodiments, may utilize electromagnets.

In certain embodiments, one or more electromagnets (not shown) may be employed in the selectively magnetizable drive head 30 in lieu of, or in addition to, permanent magnets. The electromagnet may include one or more solenoids having a ferromagnetic core that substantially align with at least one ferrous connector 24 of the magnetically driven impeller 20. The flow of current to the solenoids may be terminated to turn off the magnet for coupling/decoupling and then turned back on for use of the impeller. As will be appreciated, the current may be DC or AC and the source may be a battery or outlet. The strength of the current may vary depending upon the agitation torque/RPM requirements for the vessel. In an embodiment, the electromagnets may be located within a drive head surrounding a rotatable drive shaft. In use, the drive head and shaft would rotate to rotate an impeller.

Referring back to FIG. 5, an exemplary drive head 30 includes a first base portion 32 and a second base portion 38 that are rotatable about drive shaft 36. In embodiments, the base portions 32, 38 are manufactured from a non-magnetic material, such as a plastic or non-magnetic metal. While the drive head 30 and impeller 20 are shown having a substantially annular profiles, other shapes, sizes, and proportions are possible without departing from the scope of the invention.

As shown, the first base portion 32 and the second base portion 38 each have a plurality of ferrous connectors 34 equidistantly surrounding apertures 31, 41 that accommodate the drive shaft 36. Each of the first base portion 32 and the second base portion 38 also includes permanent magnets 40 extending radially from the drive shaft 36 to each ferrous connector 34. As described in greater detail below, the base portions 32, 38 may be rotated relative to one another to magnetize/demagnetize the ferrous connectors 34.

Rotation of the base portions 32, 38 relative to one another aligns the ferrous connectors 34 to create a magnetic field through the ferrous connectors 34 and the permanent magnets 40. More specifically, rotating the first base portion 32 and the second base portion 38 of the drive head 30 such that the ferrous connectors 34 are aligned but the magnets 40 are oriented in opposite directions, as illustrated in FIG. 9, demagnetizes the drive head 30 by containing the magnetic field to within the drive head 30. As a result, there is no longer a magnetic force pulling the ferrous connectors 24 of the impeller 20 towards the ferrous connectors 34 of the drive head 30, allowing the impeller 20 to be removed.

This is accomplished by having adjacent magnets 40 within each of the first base portion 32 and the second base portion 38 with polarities arranged in opposite directions. In the depicted embodiment, half of the magnets 40 are aligned with a positive polarity end adjacent to the apertures that receive the ferrous connectors 34 (See, the upper magnet 40 depicted in FIG. 9) and half of the magnets are aligned with the positive polarity end adjacent to the aperture that receives the drive shaft 36 (See, the lower magnet 40 depicted in FIG. 9).

This arrangement of the polarity of the magnets 40 allows a user to alternate between the states depicted in FIGS. 8 and 9 each time the ferrous connectors 34 align as the first base portion 32 rotates relative to the second base portion 38. In the depicted embodiment, the ferrous connectors 34 align each time the first base portion 32 rotates about 60° relative to the second base portion 38.

In the embodiment depicted in FIGS. 5-9, the drive shaft 36 is retained within a central aperture 41 of the second base portion 38 and the drive shaft 36 is received in a central aperture 31 of the first base portion 32. The drive shaft 36 aligns the first base portion 32 and the second base portion 38 while also allowing rotation about a longitudinal axis of the drive shaft 36. In some embodiments, the materials of the shafts 26, 36 are ferromagnetic, allowing the shafts 26, 36 to contribute to the magnetic fields generated therein.

In a specific embodiment, the shaft 36 is ferromagnetic and is held in place within the apertures 31, 41 via the magnets 40. As will be appreciated, the shaft 36 is held with sufficient force to allow the base portions 32, 38 to rotate with the shaft 36 when it is rotated via the drive motor, while permitting the first base portion 32 to rotate about the second base portion 38 to selectively magnetize the drive head 30 for impeller coupling/decoupling. In certain embodiments, the central aperture 41 of the second base portion 38 may be a blind bore terminating in a wall or surface that provides a stop for the lower end of the shaft 36 to contact. Alternatively, the aperture 41 may include a non-magnetic surface or structure that contacts the lower end of the shaft 36. Similarly, the first base portion 32 may have a blind bore or a non-magnetic surface or structure that contacts the upper end of the shaft 36 to prevent vertical movement.

In use, the drive head 30 in a demagnetized state may be moved into position under the impeller 20 via horizontal movement, as opposed to being raised into place via lifting equipment. The drive head 30 may then be magnetized to couple the head to the impeller. At this point, the motor may be activated to rotate the shaft 36 which rotates the base portions 32, 38 of the drive head and, in turn, the magnetically coupled impeller 20 to agitate fluid in the vessel. Once agitation is complete, the motor is turned off and the drive head 30 is demagnetized by rotating the first base portion 32 relative to the second base portion 38. The drive motor may then be moved away from the impeller 20.

In embodiments, rotation of the first base portion 32 may be accomplished by hand via insertion of a tool, e.g., a rod or the like, into a circumferential aperture 42 of the first base portion 32 and then urging the first base portion 32 to rotate about the shaft 36, e.g., about 60 degrees, until the drive head is in a demagnetized state. In other embodiments, rotation of the first base portion 32 may be accomplished by a motor or a lever.

Referring back to FIGS. 5-7, in certain embodiments, the base portions 32, 38 also contain additional alignment guide members in the form of an alignment receiver 33 and a slot-like alignment guide 37 containing a track 39. The alignment receiver 33 extends from an outer circumferential surface of the first base portion 32 and provides an aperture that receives a fastener, e.g., a bolt or pin (not shown), which extends through the aperture and into the track 39 of the alignment guide 37.

As shown, the alignment guide 37 extends from an outer circumferential surface of the second base portion 38 and defines the track 39 that receives the fastener while also allowing rotational movement of the fastener about the longitudinal axis of the drive shaft 36. The track 39 limits the rotational movement of the fastener to an operating angle A. In the depicted embodiment, the operating angle A is between about 60° and 120°, measured about a longitudinal axis of the drive shaft 36. In other words, the fastener, the alignment receiver 33, and the alignment guide 37 limit rotational movement of the first base portion 32 relative to the second base portion 38 to the operating angle to magnetize or demagnetize the drive head.

In the embodiment depicted in FIGS. 5 and 6, the first base portion 32 has two alignment receivers 33 arranged on opposite sides and the second base portion 38 has two complementary alignment guides 37 arranged on opposite sides. As will be appreciated, however, embodiments may include greater or fewer receivers 33 and guides 37 without departing from the scope of the invention.

In another embodiment of the invention, a method for coupling the impeller 20 of a vessel to a drive motor 30 is provided. The method includes placing a drive head 30 of the drive motor in proximity to an impeller 20 that is located within an interior volume of the bag 15. As illustrated in FIG. 8, the drive head 30 is in a demagnetized state and the impeller 20 includes a ferrous impeller connector 24. Then the method includes magnetizing the drive head 30 to create a magnetic bond between a drive head connector 34 of the drive motor and a ferrous impeller connector 24 as illustrated in FIG. 9. The demagnetized state of the drive head 30 prevents the magnetic field from emanating externally by closing the circuit within the drive head 30. In embodiments, the ferrous connector 34 is not a permanent magnet.

In one embodiment, a rotatably magnetizable drive head configuration may be employed. Here, moving from the magnetized state of FIG. 8 to the demagnetized state of FIG. 9 requires rotation of the first base portion 32 relative to the second base portion 38 such that the ferrous connectors 34 in each base portion align with one another about the drive shaft 36 and the magnets 40 in the first base portion 32 and the second base portion 38 are oriented in the same direction. When the ferrous connectors 34 in the first base portion 32 and the second base portion 38 are aligned and the polarity of the magnets 40 are oriented in the same direction, as illustrated in FIG. 8 the ferrous connectors 34 generate a magnetic force to drawing the ferrous connectors 24 of the impeller 20 towards the drive head 30. When the drive head 30 is in the magnetized state illustrated in FIG. 8, the impeller 20 rotates with the drive head 30. In the depicted embodiment, the first base portion 32 and the second base portion 38 each have six (6) magnets 40. An alternative number or arrangement of magnets 40 within the drive head 30 do not depart from the scope of the present invention.

In other embodiments, it may be possible to utilize other non-rotatable magnetizing mechanisms, e.g., a slidable apparatus. In yet other embodiments, and as described above, an electromagnet may be utilized to selectively magnetize the drive head. In such embodiments, current would be applied or removed to a solenoid within the base portion to selectively magnetize the drive head for coupling/decoupling with the impeller.

In yet another embodiment of the invention, a method of agitating fluid in a bag 15 is provided. The method includes rotating a drive head 30 of a drive motor to rotate an impeller 20 located within an interior volume of the vessel. Rotation of the impeller 20 is accomplished via a magnetic bond between a selectively magnetizable drive head connector 34 of the drive motor and a ferrous connector 24 of the impeller 20. In embodiments, the connectors 24, 34 are not permanent magnets.

In an additional embodiment, a method of decoupling an impeller of a vessel from a drive motor is provided. The method includes demagnetizing a drive head 30 of a drive motor via, for example, rotation of the first base portion 32 as described above, to remove a magnetic bond between a drive head connector 34 of the drive motor and a ferrous impeller connector 24 of an impeller 20 located within an interior volume of the vessel.

Embodiments of the impeller 20 and drive head 30 provide numerous benefits over existing impellers 100. First, the drive head 30 gives the opportunity to increase the holding torque by increasing the number of magnets 40 in the drive head 30. As a result, the drive head 30 has increased holding torque and can accommodate increased rotations per minute over those known in the art. Next, the impeller 20 and the drive head 30 have relatively few and simple parts, decreasing the costs of the hardware necessary to bring the impeller into contact with the drive head and related maintenance. Additionally, the ferrous connectors 24 in the impeller 20 are lower cost than permanent magnets. This results in an impeller 20 that is cheaper to replace. Finally, there is no vertical movement in the drive head 30 or impeller 20. As a result, a close gap between the ferrous connectors 24 and the ferrous connectors 34 can be achieved further increasing torque and speed vs known drive heads/impellers.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1-15. (canceled)

16. A vessel comprising:
an interior volume configured to contain a liquid;
a magnetically driven impeller located within the interior volume, the impeller including a rotatable base portion with at least one blade, a rotatable shaft, and at least one ferrous connector; and
wherein the impeller may be coupled to an external motor, and rotated to agitate liquid in the interior volume, via a magnetic bond between the at least one ferrous connector and a selectively magnetizable drive head connector of the external motor; and
wherein the at least one ferrous connector is not a permanent magnet.

17. The vessel of claim 1 wherein the magnetically driven impeller does not contain any permanent magnets.

18. The vessel of claim 1, wherein the at least one ferrous connector is a plurality of ferrous connectors formed in the base portion of the impeller, the ferrous connectors being spaced apart about the rotating shaft.

19. The vessel of claim 1, wherein the at least one ferrous connector is a steel connector or a plurality of steel cylinders.

20. The vessel of claim 1, wherein the vessel is a bioreactor bag.

21. A method for coupling an impeller of a vessel to a drive motor comprising:
placing a drive head of the drive motor in proximity to an impeller that is located within an interior volume of the vessel, the drive head being in a demagnetized state and the impeller including at least one ferrous impeller connector; and
magnetizing the drive head to create a magnetic bond between a drive head connector of the drive motor and a ferrous impeller connector.

22. The method of claim 21, wherein the ferrous impeller connector is not a permanent magnet.

23. The method of claim 21, further comprising:
demagnetizing the drive head so that there is no longer a magnetic bond between the drive head connector and the ferrous impeller connector; and
removing the drive motor from the impeller.

24. The method of claim 21, further comprising:
rotating the drive head to rotate the impeller via the magnetic bond between the drive head connector and the ferrous impeller connector.

25. The method of claim 21 wherein the impeller does not contain any permanent magnets.

26. The method of claim 21, wherein the at least one ferrous impeller connector is a steel connector and wherein the vessel is a bioreactor bag.

27. A method of agitating fluid in a vessel comprising:
rotating a drive head of a drive motor to rotate an impeller located within an interior volume of the vessel; and
wherein rotation of the impeller is accomplished via a magnetic bond between a selectively magnetizable drive head connector of the drive motor and a ferrous connector of the impeller.

28. The method of claim 27, wherein the ferrous connector is not a permanent magnet.

29. The method of claim 27, wherein the impeller does not contain any permanent magnets.

30. The method of claim 27, wherein the ferrous connector is a steel connector and the vessel is a bioreactor bag.
